(19) United States
(12) Patent Application Publication
(10) Pub. No.: US 2018/0008653 A1
(43) Pub. Date: Jan. 11, 2018

(54) A METHOD OF PREVENTING INFECTION OF HYMENOPTEROUS INSECTS OF THE SUPERFAMILY APOIDEA

(71) Applicant: The Marine Biological Association of the United Kingdom, Plymouth (GB)

(72) Inventors: Declan SCHROEDER, Plymouth (GB); Gideon MORDECAI, Plymouth (GB)

(21) Appl. No.: 15/539,144

(22) PCT Filed: Dec. 22, 2015
(86) PCT No.: PCT/GB2015/054136

§ 371 Date: Jun. 23, 2017

(30) Foreign Application Priority Data
Dec. 23, 2014 (GB) 1423113.8

(51) Int. Cl. A61K 35/76; C12N 15/79

(52) U.S. Cl. CPC A61K 35/76; C12N 15/79

(57) ABSTRACT

A method of preventing infection of a hymenopterous insect of the superfamily Apoidea comprising the application of an avirulent virus form thereto.

The present invention relates generally to the protection of insects against viral infection and particularly to a method of preventing infection of hymenopterous insects of the superfamily Apoidea.

The present invention is based around studies indicating that naturally occurring superinfection exclusion in honey bees explains long-term survival of Varroa infested colonies.

Over the past 50 years, millions of managed and feral honey bee (Apis mellifera) colonies have died as the ecto-parasitic mite Varroa destructor has spread into the West. The mite has introduced a new viral transmission route that has dramatically altered the viral landscape. This has resulted in a massive loss of diversity in Deformed Wing Virus (DWV), the pathogen now linked with the world-wide collapse of honeybees colonies. However, before Varroa spread, DWV co-existed with honey bees albeit at viral loads many orders of magnitude lower than is now observed. History of introduced pathogens tells us that honey bees will either succumb to this new imbalance as demonstrated by the decimation of feral honeybee populations or evolve new equilibriums with this parasite-virus association⁷. In the West, very few and often isolated A. mellifera populations infested with Varroa are known to persist without direct human intervention, and even then control mechanisms remain rudimentary and largely unpredictable in terms of outcome. We discovered a phenomenon known as Superinfection exclusion (SIE) among DWV strains that explains why one such isolated UK A. mellifera population has survived for nearly two decades, despite high Varroa and DWV loads. That is a non-lethal variant of DWV (type B) has become established in both A. mellifera and Varroa populations thus preventing secondary infections of the lethal ‘type A’ DWV variant becoming established. This appears to be achieved by recombining out the lethal variant; a host-pathogen relationship which, notwithstanding major differences in the immune systems of bees and humans, bears a striking resemblance to Edward Jenner's 18th century discovery that pre-exposure to the relatively innocuous cowpox virus went on to protect milkmaids against the deadly smallpox virus. Building on this original vaccination example, this novel bee virus-interaction would seem to hold great promise for the development of an effective prophylactic treatment for use in the worldwide battle against this highly destructive virus.

The recent global decline of the European honey bee (Apis mellifera) populations (Ratnieks & Carreck, 2010; Schroeder & Martin, 2012) is of grave concern due to their role as pollinators which contribute an estimated $225 billion to the global economy (Gallai et al, 2009). For over half a century the global spread of the ectoparasitic mite, Varroa destructor, has resulted in the death of many millions of managed and feral honey bee colonies (Martin et al, 2012; Schroeder & Martin, 2012; Thompson et al, 2014). The mite has introduced a new viral transmission route that has dramatically altered the viral landscape (Martin et al, 2012). This has resulted in a massive loss of diversity in Deformed Wing Virus (DWV) (Martin et al, 2012), the pathogen now linked with the collapse of honey bee colonies (Di Prisco et al, 2011; Highfield et al, 2009). However, prior to Varroa spread, DWV stably co-existed with honey bees (Martin et al, 2012) albeit at viral loads many orders of magnitude lower than is now observed (Martin et al, 2012; Mondet et al, 2014). For example, the recent arrival of Varroa into the Hawaiian honey bee population was accompanied by a million fold increase in the viral load of DWV, loss of DWV diversity and the predominance of a single highly virulent DWV variant (type A) (Martin et al, 2012). These landscape scale changes have also been demonstrated at the individual honey bee level within the UK honey bee population. For example, Ryabov et al (2014) demonstrated the dominance of a single variant of DWV when a mixture of viral strains were injected into developing pupae leading to a rapid loss of DWV diversity and million fold increase in viral loads.

DWV is a rapidly evolving group of closely related variants (de Miranda & Genersch, 2010), commonly referred to as a quasispecies (Domingo & Holland, 1997; Lauring & Andino, 2010). Within the DWV quasispecies there are several master variants, each with its own swarm of variants. Each variant can form potential recombinants with other variants, within a swarm and between master variants. Kakugo virus (KV) is a variant of the DWV type A that differs from the master sequence (Lanzi et al, 2006) by 6% in the non-structural coding region (Baker & Schroeder, 2008; Fujiyuki et al, 2006), whereas, Varroa destructor Virus-1 (VDV-1) (Ongus et al, 2004), is genetically dissimilar to DWV type A (84% genome identity) and is referred to as DWV type B (Martin et al, 2012). Notably, both DWV type A and B master variants are able to replicate within mites and honey bees, and both have been detected in honey bees in the absence of Varroa (Martin et al, 2012; Yue & Genersch, 2005; Zioni et al, 2011). Recombinants between the variants have been reported (Moore et al, 2011; Ryabov et al, 2014; Zioni et al, 2011) and a novel recombinant between DWV type A and a new DWV master variant, type C, has also been recently discovered (Mordecai et al, 2015), suggesting that they are part of the same quasispecies and share a recent common ancestor. DWV type A has been detected in honey bee populations around the world and in the presence of Varroa leads to colony death (Di Prisco et al, 2011; Martin et al, 2012), whereas there are no known instances of type B being linked to colony death. The role of the new type C in overwintering colony losses is currently unclear (Highfield et al, 2009; Mordecai et al, 2015).

In the early 1990s, Varroa swept across the UK and was followed by widespread colony deaths 1 to 3 years later. To ensure the long-term survival of their honey bee colonies, beekeepers in Varroa infested countries manage Varroa populations (Sumpter & Martin, 2004), largely through chemical methods. Nonetheless, there are reports of rare isolated untreated A. mellifera colonies of European origin thriving despite Varroa infestation including cases on an island in Brazil (DeJong & Soares, 1997), and in small forest patches in France (Conte et al, 2007) and New York, USA (Seeley, 2007). The survival of these colonies is well documented and not questioned, however, the mechanism by which tolerance to Varroa and its association with DWV is maintained remains elusive. In the UK a small number of beekeepers opted not to control their mite populations and in most cases lost their bees. However one UK beekeeper, Ron Hoskins, initiated a closed breeding program from colonies that survived the initial Varroa infestation and this isolated population of up to 40 colonies persists in Swindon, central England, without chemical control of Varroa (http://www.swindonhoneybeeconservation.org.uk/).

The present invention is based on a study which assessed the viral landscape in this apiary thereby determining whether the colonies remained disease free due to an absence of DWV. The present inventors have shown the Swindon apiary is dominated by an avirulent DWV type B master variant with the concomitant absence of the virulent DWV type A master variant. Taken together, this data suggest that a phenomenon known as superinfection exclusion is the explanation for why this isolated UK honey bee population has survived, despite Varroa infestation and high DWV loads.

According to an aspect of the present invention there is provided a method of preventing infection of a hymenopterous insect of the superfamily Apoidea comprising the application of an avirulent virus form thereto.

The present invention may therefore relate to a non-natural intervention to inoculate one, some or substantially all members of a bee colony using a superinfection exclusion principle.

The present invention may be applicable to any hymenopterous insect of the superfamily Apoidea, which includes social forms such as the honeybee and solitary forms such as the carpenter bee, bumblebees and mason bees.

The present invention also provides a method of preventing infection of a bee by a virulent virus variant comprising inoculating with an avirulent virus form.

The present invention also relates to use of an avirulent virus form for the manufacture of an inoculum for a bee.

The present invention also relates to use of an avirulent virus form for the treatment or prevention of a virulent virus infection of a bee.

The present invention also relates to use of an avirulent virus form for the prevention of premature death of individual bees or a bee colony as a whole.

The present invention also relates to a method of superinfection exclusion protection of bees comprising inoculation with an avirulent virus form.

The insect/bee may be from the family Apidae. For example the insect/bee may be from the genus Apis. Alternatively or additionally the insect/bee may be from the genus Bombus. Alternatively or additionally the insect/bee may be of the species Apis mellifera.

In some aspects and embodiments the virulent virus may be deformed wing virus (DWV). For example the virulent virus may be type A DWV.

The avirulent virus form may be naturally occurring and/or recombinant and/or mutated or variant forms thereof.

The avirulent virus form may be type B DWV.

The avirulent virus form may be type C DWV.

The present invention also provides use of type B DWV or a variant or mutant form thereof for the treatment or prevention of type A DWV infection of Apis mellifera.

The present invention also provides a method of preventing infection of Apis mellifera by type A DWV comprising inoculating with type B DWV or a variant or mutant form thereof.

The present invention also provides use of type B DWV of a variant or mutant form thereof for the manufacture of an inoculum for Apis mellifera.

In aspects and embodiments of the present invention the avirulent form may be administered by: topical application; immersion or spraying; contamination; or through a food (natural or synthetic); by injection; or by introduction of a vector (for example infected mites such as Varroa destructor mites).

The present invention also provides a composition comprising an effective dose of an avirulent viral form and a suitable carrier or excipient.

The present invention also provides an inoculum formulation comprising an effective dose of infectious an avirulent viral form and a suitable carrier or excipient.

The present invention also provides a composition comprising an effective dose of infectious type B DWV or a variant or mutant thereof and a suitable carrier or excipient.

The present invention also provides an inoculum formulation comprising an effective dose of infectious type B DWV or a variant or mutant thereof and a suitable carrier or excipient.

Different aspects and embodiments of the invention may be used separately or together.

The present invention is more particularly shown and described, by way of example, with reference to the accompanying drawings, in which:

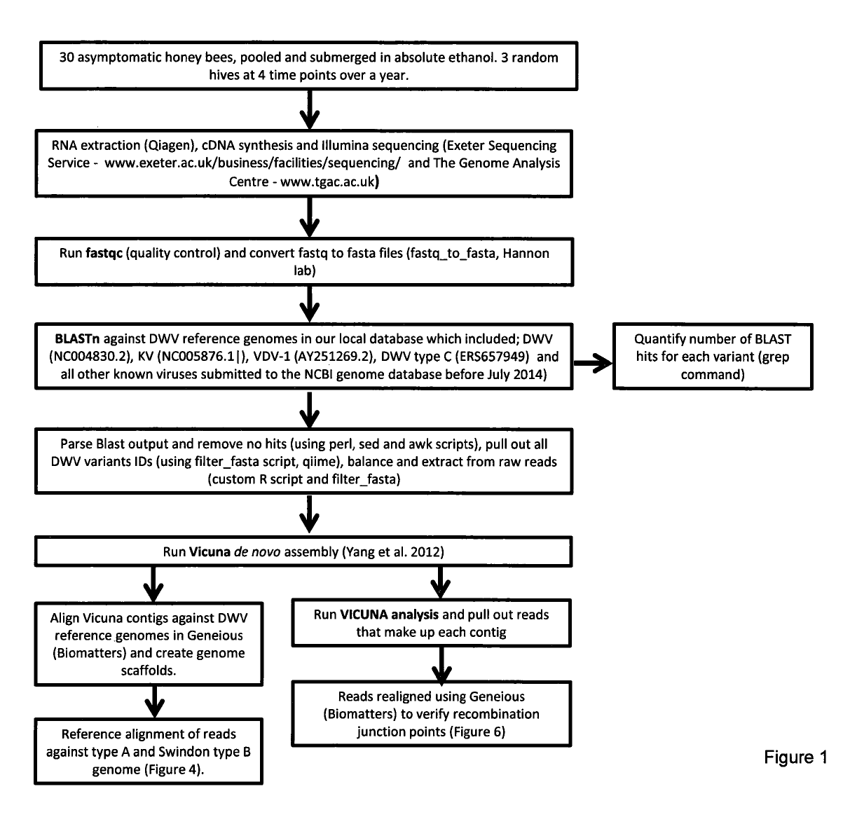

FIG. 1. Bioinformatics pipeline leading to the application of the Vicuna de novo assembler.

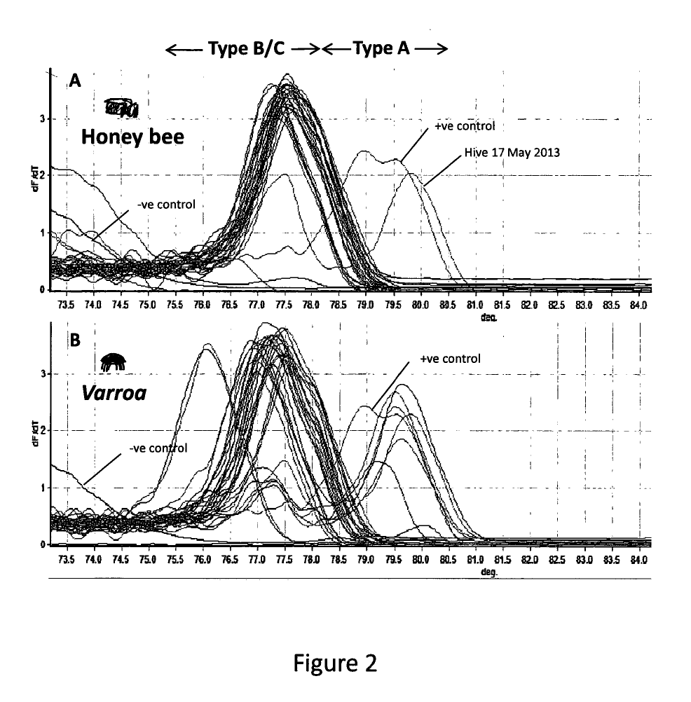

FIG. 2. High resolution melt (HRM) curve analysis for DWV RdRp RT-qPCR region for three hives in the UK colonies (hive 6—blue, hive 17—cyan, hive 19—Green) a, Honeybees and b, Varroa mites distinguishing between DWV type A and B/C variants. Deformed winged symptomatic bees were used as a positive control (pink line). A no template negative control was also run (black line).

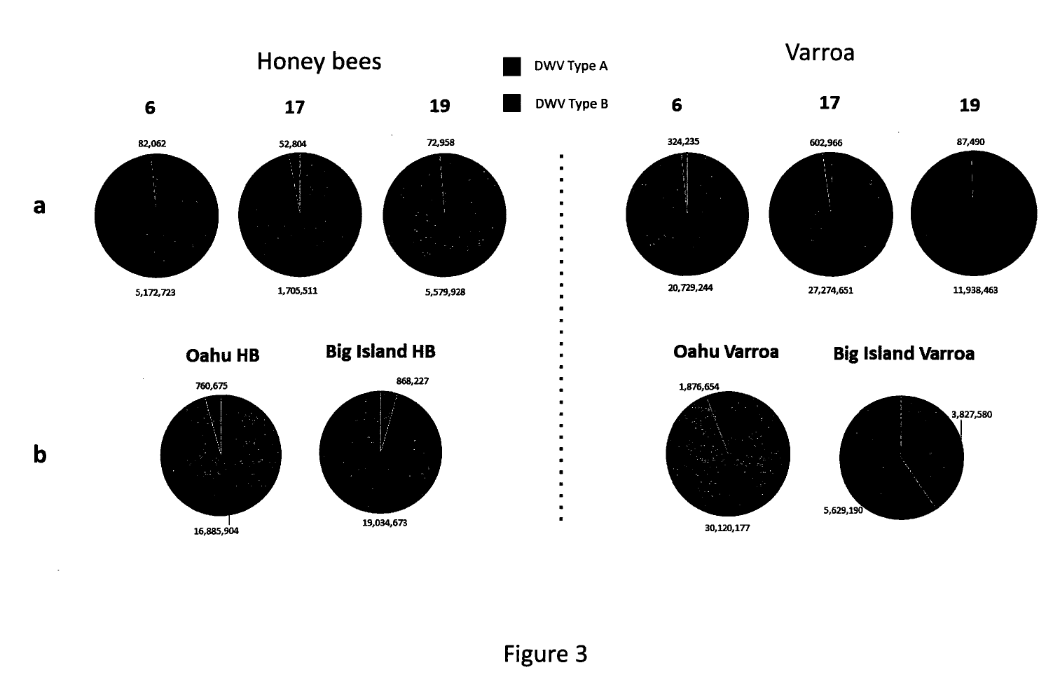

FIG. 3. Proportions of DWV subgroups within colonies sequenced using Illumina Hi-seq. a, Swindon samples collapsed into their respective hives 6, 17, and 19. b, the Hawaiian samples from Oahu and Big Islands. A BLASTn algorithm against a custom DWV quasi-species database was used and the numbers indicate of hits to each DWV variant.

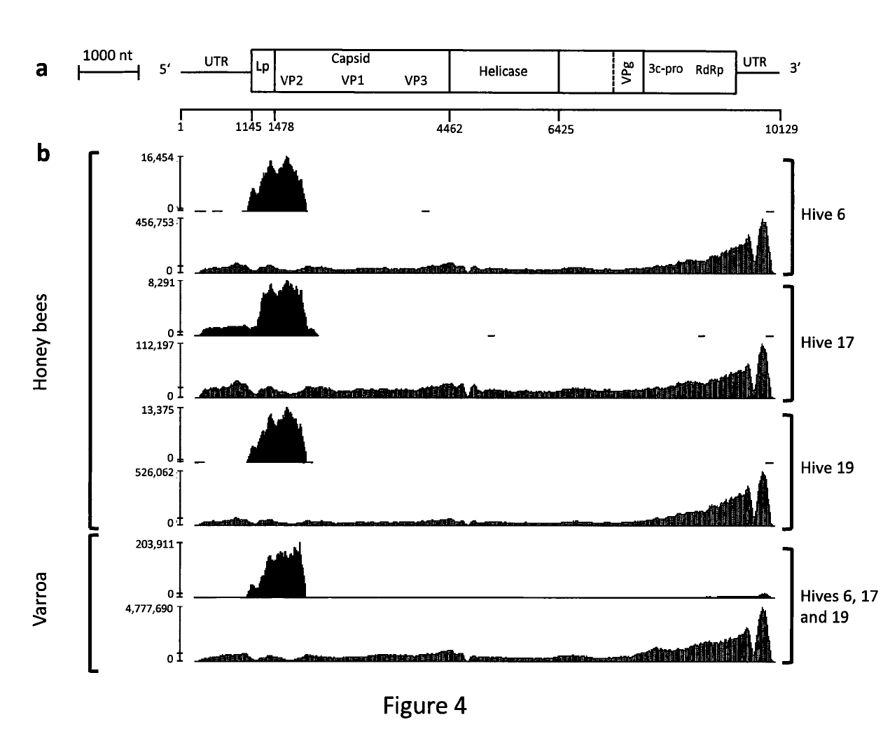

FIG. 4. Genome coverage from the Illumina Hi-seq data for the Swindon colonies. a, Map of the DWV genome adapted from Lanzi et al. 2006. b, DWV type A and B genomes (in red and blue, respectively) assembled from the Illumina NGS data from honeybees and mites from the Swindon apiary (hives 6, 17 and 19). De novo assembled VICUNA contigs that makeup these genomes for each hive were deposited in European Nucleotide Archive (ENA) under accession numbers ERS636096 to ERS636117.

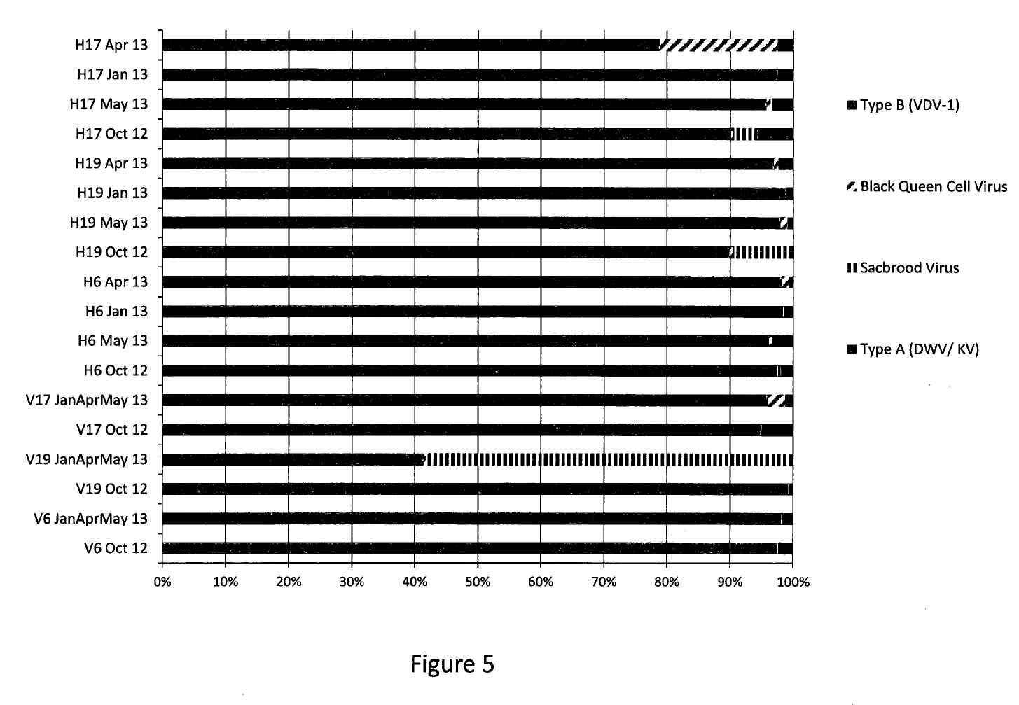

FIG. 5. Virome of the Swindon apiary. Illumina reads were searched against a viral database (FIG. 1) using BLASTn and the proportion of top hits associated with honeybee viruses was counted. DWV type B dominated the monthly samples in both the honeybee samples (H6, H17, H19) and the Varroa samples (V6, V17, V19).

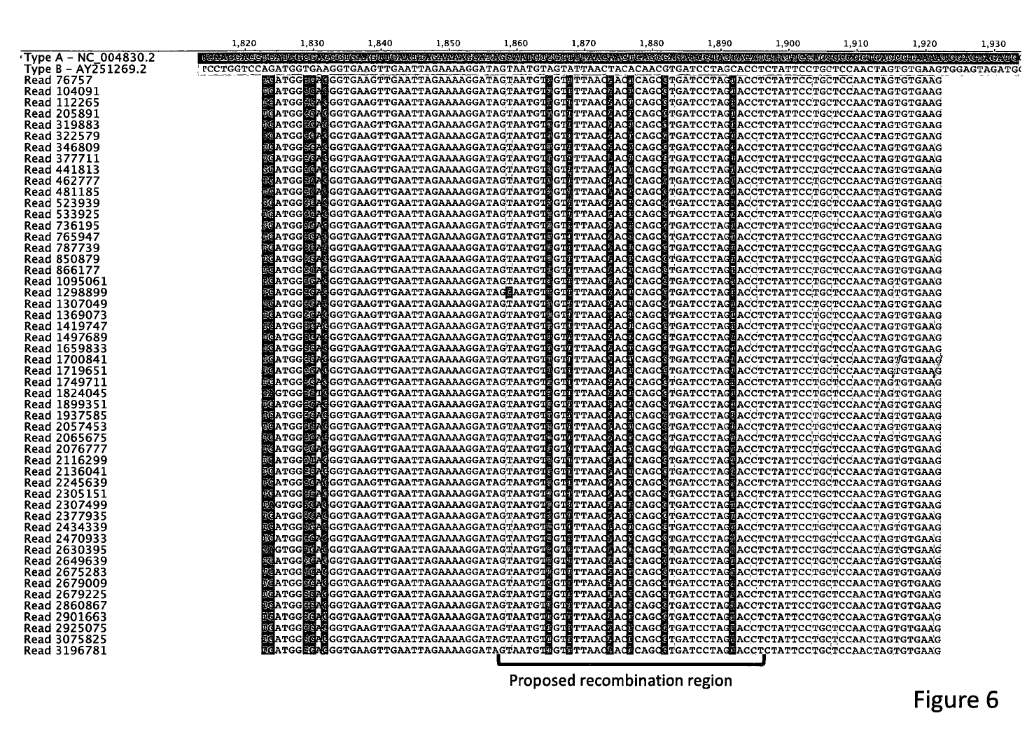

FIG. 6. A multiple sequence alignment of the reads covering the recombination junction in the DWV recombinat from H19, April 2013. Output from Vicuna analysis was converted into a suitable format and imported into Geneious to visualise the reads over the type A-B recombination junction point. The DWV type A and B reference sequences are shown at the top and highlighted red and blue respectively. Base pair substitutions common to either DWV type A and B variants are highlighted in each 100 bp Illumina read. In this example, 52 out of a total 2464 reads is shown that covers the proposed recombination region

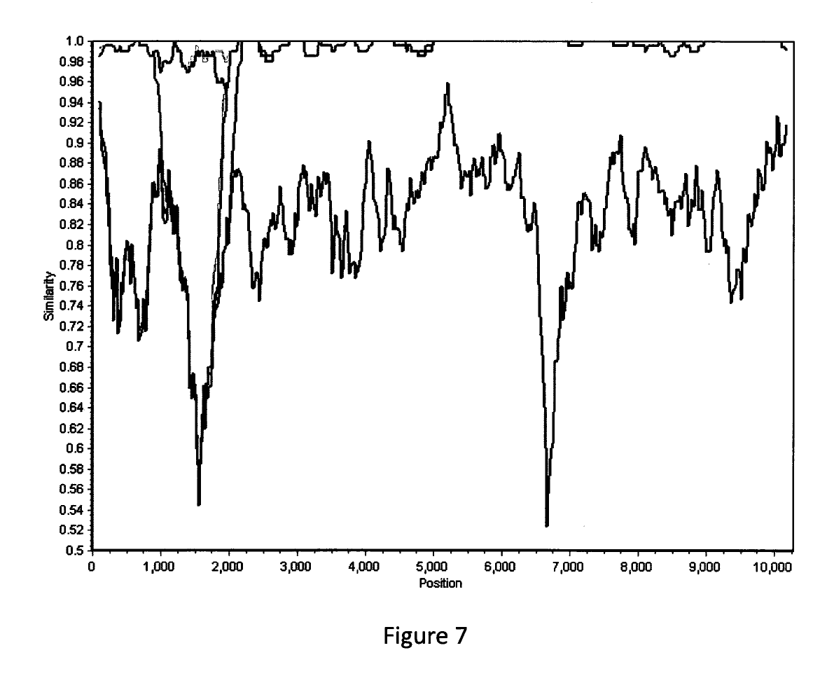

FIG. 7. Simplot analyses of the different genomes present in the Swindon samples. Nucleotide similarities of various variants are compared to the Type B (VDV) reference genome (AY251269.2). The type A (DWV) reference genome (NC004830.2) is shown in red. A selection of DWV genome scaffolds containing recombination in the 5′ end of the genome are shown; neon and dark green (type B-A-B recombinant from January 2013 Hives 17 and 6, respectively), cyan (Swindon type B genome from Hive 6 Jan. 2013), dark blue (Swindon type B genome from Hive 17 Jan. 2013) and pink (type A-B recombinant, H17 Apr. 2013). A sliding window of 200 nt was used, moving in a step of 20 nt.

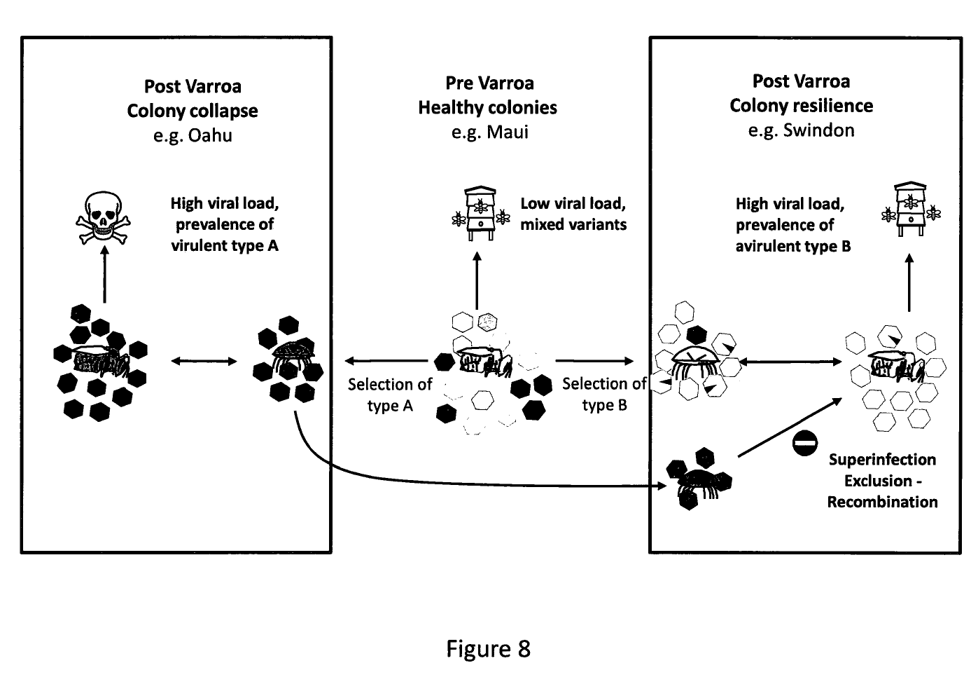

FIG. 8. New honeybee-Varroa mite-DWV equilibrium. Type A DWV is represented in red and type B in blue. In Varroa free hives DWV exists as a cloud of variants present at low levels. In diseased hives such as Oahu, the type A is present in a Varroa mediated transmission cycle. Whereas in Swindon, transmission of type B between bees and Varroa prevents the incursion of the type A variant into honeybees and consequently the hive survives.

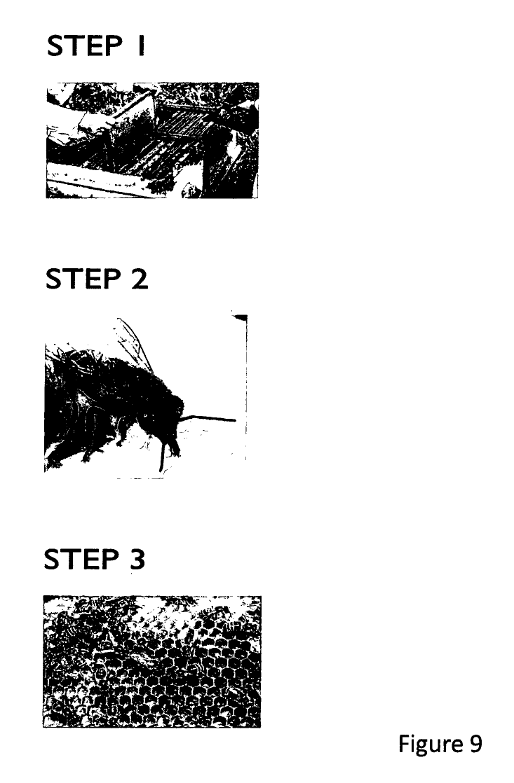

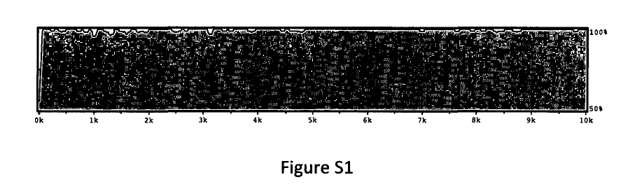

FIG. 9. A schematic representation of a method of bee superinfection exclusion inoculation conducted in accordance with the present invention.

### SUPPLEMENTARY FIGURE

Figure S1. Plot showing the percentage identity across the whole genome of the genome scaffold from Hive 6 Jan. 2013 (ERS754547) compared to the type B VDV reference genome (AY251269.2). The two genomes are 99.5% identical.

Example embodiments are described below in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included. Elements of the example embodiments are consistently denoted by the same reference numerals throughout the drawings and detailed description where appropriate.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

Material and Methods:

Sample Collection

Pooled asymptomatic honey bees were collected from sites in Hawaii and from the Swindon Apiary. A time series of three hives in Swindon was taken (10 time points over a year, 4 of which were used for Illumina sequencing per hive). Varroa samples were collected from the same three hives in the Swindon apiary alongside the honey bee samples.

DWV Detection Assay

RNA extractions, RT-PCR and High Resolution Melt (HRM) analysis were carried out according to a previous study (Martin et al, 2012). In brief, total RNA was extracted from whole worker honey bees using an RNeasy mini kit (Qiagen), according to the manufacturer's instructions. RT-PCR via oligo dT priming using previously designed DWV primers (Highfield et al, 2009) and subsequent HRM was carried out using Sensifast No-Rox One Step Kit (Bioline). RNA was diluted to ˜100 ng/μl and 1 μl of template RNA was added per reaction. The DWV load per worker honey bee was calculated according to the method developed by Highfield et al (2009). The amount of RNA used for each RT-PCR reaction was normalised per bee and the DWV load per bee was calculated through a DWV cRNA standard curve conversion (y=−3.695x+32.744).

Bioinformatics Pipeline

Illumina Hi-seq (2×100 bp) pair-end sequencing was carried out by The Genome Analysis Centre (TGAC) and the University of Exeter. Total RNA was sequenced after a cDNA synthesis step with no amplification step necessary. Varroa RNA was pooled for 3 of the time points (January, April and May) prior to Illumina sequencing. A bioinformatics pipeline (FIG. 1) was developed to accommodate the large amount of variation found within the DWV species complex. Firstly, the quality of the raw reads was verified using FastQC (Babraham bioinformatics). Samples were then converted from fastq to fasta using the fastq_to_fasta script which is part of the FASTX-toolkit (Hannonlab, http://hannonlab.cshl.edu/fastx_toolkit/). To isolate the reads sequenced from the DWV complex from the host and other contaminating sequences the BLASTn tool was used (Altschul et al, 1990). The reads were searched against a custom BLAST database containing the DWV, VDV-1 and KV genomes, with an e value of 10e-05. BLAST was carried out against Read 1 of the Illumina data. The ncbiblastparser perl script (http://www.bioinformatics-made-simple.com/2012/07/ncbi-blast-parser-extract-query-and.html) was then used to parse and read the top hit of the BLAST output. Next, ‘sed’ and ‘awk’ scripts were used to delete empty lines and the reads, which contained ‘nohits’. The corresponding BLAST hits were then pulled out from the Read 2 raw reads using QIIME. The paired reads were then balanced using a custom script written in R version 3.2.0 (R Core Team, 2015). which deletes any read in the Read 1 BLAST top hit file which did not have a pair in the corresponding read 2 BLAST top hit file and vice versa. The balanced DWV family reads were then assembled using the Vicuna assembler, which was developed to generate consensus assemblies from genetically heterogeneous populations, specifically RNA viruses (Yang et al, 2012).

Vicuna contigs greater than 300 bp in length were imported into Geneious (Version 7.04, created by Biomatters) and the ‘Map to Reference tool’ was used to align the contigs with the Type A and B reference genomes. For several of the samples the Vicuna assembly yielded full-length contigs that covered the whole genome, whilst others yielded only several smaller contigs (Table S1). The ends of the contigs were then edited to remove discernable assembly or sequencing artefacts. Assembled DWV contigs were uploaded to the European Nucleotide Archive under the Study accession PRJEB8112. Vicuna contigs from hive 6 Jan. 2013 were used to assemble a type B variant genome (accession number ERS754547).

The identity of the type B genome was compared to the VDV reference genome using the mVista tool (Figure S1) (Frazer et al, 2004), and the phylogeny of the Swindon variant was determined from a neighbor-joining tree of the polyprotein encoding region of the DWV genome (Lanzi et al, 2006). Additionally, genome scaffolding was carried out to produce full length genomes representing the unique recombinant present in Swindon. SimPlot software was used to visualise the recombination event (Lole et al, 1999).

To investigate the genome coverage of each DWV variant in Swindon, reads were grouped per hive (Varroa samples were all grouped together) and competitively aligned against the type A reference genome (NC 004830.2) and the Swindon type B genome using the Geneious map to reference tool. The maximum percentage of mismatches per read accepted was 5% and no gaps per read were allowed.

To examine individual reads that make up the consensus sequence of each contig the Vicuna analysis tool was used. In order to view the reads in Geneious the Vicuna analysis output was modified by using a sed script to keep just the sequence reads. These were then converted from a tabular format into a fasta format using the python script ‘tab2fasta.py’ and then visualised using Geneious. To quantify the number of reads with sequences similar to either DWV variant (type A or B) the Illumina reads were searched against a viral database using BLAST and the number of top hits attributed to each reference genome was quantified. Finally, genome coverage was calculated using the Lander/Waterman equation (read length×number of reads/genome length), which estimates the depth of sequencing across the genome (Sims et al, 2014).

Results and Discussion

Using a combination of RT-qPCR, HRM (Martin et al, 2012) and Illumina (2×100 bp) Hi-seq sequencing (FIG. 1) we investigated the DWV viral load and diversity in this small honey bee population in Swindon and their associated Varroa mites. Three hives were chosen at random and pools of 30 asymptomatic worker bees were sampled from inside the colony on 10 occasions at roughly monthly intervals between October 2012 and October 2013. RT-qPCR on an RNA dependent RNA polymerase (RdRp) gene fragment for all 30 samples collected confirmed the persistence of high DWV loads (10⁷ to 10⁸ copies per bee) during the entire study period in all three hives (Table S2). Both the DWV load and prevalence found within this study suggest that DWV presence alone cannot explain colony losses as proposed in previous Hawaiian (Martin et al, 2012) and Devon, UK, studies (Highfield et al, 2009).

To explore other factors that might contribute to this discovery, we exploited the known nucleotide polymorphisms in the RdRp gene fragment among the known DWV master variants (A, B & C) (Martin et al, 2012; Mordecai et al, 2015). HRM indicated the dominance of the type B or C master variant (FIG. 2a), as these have similar melting temperatures (Martin et al, 2012; Mordecai et al, 2015). Only a single honey bee sample out of 30 tested contained both DWV type A and B/C, suggesting that while a colony can be exposed to type A, it fails to establish and neither persists nor accumulates. In contrast to the bee samples, the Varroa samples contained a greater mix of both DWV type A and B/C (FIG. 2b), although type B/C remained the most prevalent. This prevalence of type B/C over A contrasts to what a previous showed in Hawaii where the type A master variant dominated (Martin et al, 2012) and suggests that type B/C may be an avirulent form of DWV. However, given that HRM analysis only detects limited genomic change (within the RdRp gene fragment), the possibility of recombination outside the RdRp region cannot be excluded. Both Mordecai et al (2015) and Ryabov et al (2014) showed that certain recombinants of the master variants A-C, and A-B, respectively, could be more lethal than the type A master variant.

DWV type B master variant dominance was however confirmed by Illumina sequencing (FIGS. 3-5). As a proportion of the total sequenced Illumina reads, DWV hits accounted for an average of 46.3% of reads in the Varroa samples and 9.7% of reads in the honey bees. The average DWV genome coverage for the honey bee samples was 22,484×, while the Varroa samples had an average DWV coverage of 599,558×. Vicuna assembly produced 6410 contigs across the 18 samples (Table S1). Sample “Hive 6 Jan. 2013” was used to assemble the “Swindon” DWV type B variant (Table S3) which was found to be 99.5% identical to the type B reference genome (VDV-1) (Figure S1). FIG. 4 also shows that the type B DWV coverage was high, with over fifteen million reads aligned from the honey bee samples compared to two hundred and forty one thousand reads aligned to the type A reference. Similarly, in the Varroa samples seventy one and a half million reads aligned to type B compared to just over one million for type A. Type B reads aligned across the whole genome, whilst full genome coverage of type A was restricted to the Varroa samples. No reads unique to the Devon DWV type C genome could be found, whether in the honey bee or Varroa samples. In all, the honey bee and Varroa associated virome of the isolated UK study colonies was predominantly DWV type B (FIG. 5), indicating that alternate DWV master variant competitive outcomes are possible.

De novo and reference assembly of the DWV variant genomes suggested that recombination has taken place with type A possibly being recombined out, as evidenced by the presence of DWV recombinants within the honey bee samples (FIGS. 4, 6 and 7). Full genome scaffolds of each recombinant were made using the Vicuna contigs. These were aligned with type A and B genomes and Simplot (FIG. 7) revealed that the recombination junction in the Swindon samples differed from that previously reported, Moore et al (2011) showed that a recombination junction occurred in the 5′ untranslated region of the genome whereas the Swindon DWV type A-B recombinant junction found here occurs in the structural region of the open reading frame (FIGS. 4 and 7). While full genome coverage was not achieved in both honey bee and Varroa samples by de novo assembly for type A, interestingly, reference alignment of DWV reads from the Swindon Varroa mites shows that the whole genome of type A is present at low levels (FIG. 4), although HRM analysis indicated that the type A (master or any recombinants thereof) is rapidly removed in the following five months (see FIG. 2, hive 17). A low number of type A reads (1.68% according to BLAST analysis) present in the UK study population (FIG. 3) were uniquely associated with novel recombinants (FIGS. 4, 6 and 7, Table S4) in which the majority of the genome were type B but contained a region of type A sequence at the 5′ end of the genome (the UTR and leader protein, FIG. 4). The number of reads within the region of recombination for each of the hives was counted to compare the depth of coverage between the two variants (Table S4). As this is a direct comparison of the same region of the genome, i.e. the 3′ end, which is caused by bias in reverse transcription oligo dT priming (FIG. 4), the 3′ bias is not relevant. In all hives the number of type B reads exceeded the number of type A (recombinant) reads by an order of between 4.5 (Hive 19) and 36.4 (hive 17). Therefore, the dominance of type B master variant in this UK study population appears to be correlated with a level of colony protection as it appears to exclude type A or C (and any virulent recombinants thereof).

To compare this discovery of type B dominance in this study with respect to the previous Hawaiian study (Martin et al, 2012), a small number of honey bee and Varroa Hawaiian samples with a known Varroa history were also subject to Illumina (2×100 bp) Hi-seq sequencing and analysed using the same analytical Vicuna pipeline as that used for the UK samples (FIG. 1), resulting in 212 contigs (Table S1). On Oahu, where Varroa had established and caused widespread colony death, a colony analysed by Hi-seq (173,567× coverage) revealed that type A dominated (FIG. 3b) confirming HRM data from another 28 colonies from Oahu, which also had predominantly type A (Martin et al, 2012). However, in the colony from Big Island where Varroa had been present for less than 2 years and widespread colony collapse was yet to occur, type B dominated the sequence reads (195,760× coverage). In contrast, the Varroa sample from the same colony on Big Island contained a nearly equal mix of type A and B (93,014× coverage) whereas Varroa from Oahu (314,713× coverage) was dominated by type A (FIG. 3b). A switch in dominance between type A and B in the Big Island honey bees suggests active competition between the two DWV variants consistent with the suggested 1-3 year time lag for DWV variants adapted to mite transmission to undergo selection (Martin et al, 2012). As in Swindon, no significant matches to type C could be found in the honey bee or Varroa samples on either island. The time lag of the B to A switch in Big Island corresponds to the period when the mite becomes established but before colonies start dying. The normal outcome of this variant competition is the dominance of type A as evidenced by its transmission around the world (Berényi et al, 2007). In the Varroa resistant Swindon apiary, once established the avirulent type B variant, appears to prevent type A from becoming dominant. Crucially, in Swindon the Varroa mites contained a proportion of type A reads (representing the whole type A genome) which were not detected in the honey bees suggesting that effective transmission of type A from parasite to host was prevented (FIG. 4).

Superinfection exclusion (SIE) has been well documented in viruses related to DWV, for example, Tscherne et al (2007) used cell lines to show that infection by one genotype of hepatitis C virus, prevented infection by others. SIE best explains the phenomenon of why, despite high DWV load and Varroa infestation, the isolated UK colonies do not collapse. We speculate that co-evolution of the honey bee-Varroa mite-DWV system has selected for a new stable equilibrium where both the Varroa and an avirulent type B variant of DWV protect the honey bee, and thus the colony, from the virulent type A (FIG. 8). Further work to validate this and determine the mechanism of the viral exclusion is required. For example, to demonstrate whether type B can protect against type A or C at the cellular and individual honey bee level using assays similar to those described by Ryabov et al (2014). If true, this would be the first report of SIE acting on the Iflavirus pathogens of bees. Ironically, it may be the presence of the mite population that is protecting the colony since Varroa may be providing the opportunity for constant re-introduction of type B into the population via horizontal transmission. In addition, although recombinants were present in both honey bee and Varroa samples it is unclear if these originate in the honey bees, Varroa or both.

It also remains unclear under what conditions type B can prevail or if similar mechanisms of protection operate in the Brazilian, USA and French populations. Although the mechanism for exclusion seen in the Swindon apiary is unclear, a unique recombinant between type A and B was found (FIG. 6) suggesting that the full length type A genome (FIG. 4) is actively suppressed. This is the counterpart of recombination causing acute infections as described by Moore et al (2011) and Ryabov et al (2014). Other candidate mechanisms have previously been identified in different viruses at various stages of the viral life cycle, including blocking of virus entry to the cell at the level of receptor interaction or occupation of sites for RNA replication (Lee et al, 2005). Alternatively, the dominance of type B in the Swindon samples could be due to the induction of a differential immune response from the host such as RNAi (Hunter et al, 2010).

Studies on honey bee pathogens have suggested that natural selection favours the survival and transmission of DWV over viruses of the Acute Bee Paralysis Complex (ABPV, KBV & IAPV), which have a higher virulence (Schroeder & Martin, 2012; de Miranda & Genersch, 2012). In this scenario, virus survival requires that the pupae live long enough to enable Varroa maturation and allow onward virus transmission. For example, the acute virulence of ABPV kills both adults and pupae quickly, ending the transmission cycle as mites associated with the pupae do not survive (Schroeder & Martin, 2012). The same reasoning can be applied to the DWV quasispecies where a particular host-variant dynamic dictates stable transmission or prevalence. Therefore, the Swindon UK population in question could have evolved to favour DWV type B persistence as a result of husbandry practices that have selected for a new stable non-pathogenic equilibrium. However, this phenomenon is not peculiar to Swindon as a recent study in South Africa found only DWV type B in four study apiaries, with no type A detected in either mites or honey bees (Strauss et al, 2013). This raises the possibility that SIE may be operating on a wider scale in some geographical locations.

Based on these results the present inventors have determined that within the swarm of DWV, due to SIE, different viral variants are competing with two discernible outcomes. Either the disease causing variants dominates, which can lead to colony collapse (Martin et al, 2012), or an avirulent variant can prevail, reaching high viral loads which excludes the virulent variants. In the Swindon apiary an evolutionary stable state has been reached in which disease symptoms are minimal and colonies survive. The data show that the dominance of type B in this isolated UK apiary has been stable only over a year of sampling, but anecdotal evidence suggests that the viral makeup of the bees at the Swindon Honey bee Conservation Trust has been stable for some time longer.

The development of a SIE mechanism in honey bees gives those wishing to limit or eradicate the sources of honey bee colony decline the possibility of active intervention. For example, in the citrus industry, where SIE is used to reduce crop losses by inoculating plants with a benign variant of Citrus tristeza virus to protect against infection by a more pathogenic form (Lee & Keremane, 2013). Accordingly, the direct introduction of DWV type B could provide a form of biocontrol against further collapse of European honey bee colonies in the face of Varroa infestation.

In FIG. 9 one method of protecting honey bees is illustrated. In step 1 one or more bees are isolated from a colony. In step 2 the bees are inoculated with a sugar solution containing DWV type B virus. In step 3 the inoculated bees are reintroduced into the colony.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention.

### REFERENCES

Altschul S F, Gish W, Miller W, Myers E W, Lipman D J. (1990). Basic local alignment search tool. J Mol Biol 215:403-410.
Baker A C, Schroeder D C. (2008). The use of RNA-dependent RNA polymerase for the taxonomic assignment of picorna-like viruses (order Picornavirales) infecting Apis mellifera L. populations. Virol J 5:10
Berényi O, Bakonyi T, Derakhshifar I, Köglberger H, Topolska G, Ritter W, et al. (2007). Phylogenetic analysis of Deformed Wing Virus genotypes from diverse geographic origins indicates recent global distribution of the virus. Appl Environ Microbiol 73:3605-3611.
Conte Y L, Vaublanc G D, Crauser D, Jeanne F, Rousselle J-C, Bécard J-M (2007). Honey bee colonies that have survived Varroa destructor. Apidologie 38: 566-572.
de Miranda J R, Genersch E. (2010). Deformed wing virus. J Invertebr Pathol 103 Supplement: S48-561.
DeJong D, Soares A E E. (1997). An isolated population of Italian bees that has survived Varroa jacobsoni infestation without treatment for over 12 years. Am bee J 137: 742-745
Di Prisco G, Zhang X, Pennacchio F, Caprio E, Li J, Evans J D et al. (2011). Dynamics of persistent and acute Deformed Wing Virus infections in honey bees, Apis mellifera. Viruses 3: 2425-2441.
Domingo E, Holland J J. (1997). Rna virus mutations and fitness for survival. Annu Rev Microbiol 51: 151-178.
Frazer K A, Pachter L, Poliakov A, Rubin E M, Dubchak I. (2004). VISTA: computational tools for comparative genomics. Nucleic Acids Res 32:W273-W279
Fujiyuki T, Ohka S, Takeuchi H, Ono M, Nomoto A, Kubo T. (2006). Prevalence and phylogeny of Kakugo virus, a novel insect picorna-like virus that infects the honeybee (Apis mellifera L.), under various colony conditions. J Virol 80: 11528-11538.
Gallai N, Salles J-M, Settele J, Vaissiêre B E. (2009). Economic valuation of the vulnerability of world agriculture confronted with pollinator decline. Ecol Econ 68: 810-821.
Highfield A C, El Nagar A, Mackinder L C M, Noel L M-L J, Hall M J, Martin S J, et al. (2009). Deformed Wing Virus implicated in overwintering honeybee colony losses. Appl Environ Microbiol 75:7212-7220.
Hunter W, Ellis J, vanEngelsdorp D, Hayes J, Westervelt D, Glick E, et al. (2010). Large-scale field application of RNAi technology reducing Israeli Acute Paralysis Virus disease in honey bees (Apis mellifera, Hymenoptera: Apidae). PLoS Pathog 6:e1001160.
Labrie S J, Samson J E, Moineau S. (2010). Bacteriophage resistance mechanisms. Nature Rev Microbiol 8:317-327
Lanzi G, de Miranda J R, Boniotti M B, Cameron C E, Lavazza A, Capucci L et al. (2006). Molecular and biological characterization of Deformed Wing Virus of honeybees (Apis mellifera L.). J Virol 80: 4998-5009.
Lauring A S, Andino R. (2010). Quasispecies theory and the behavior of RNA viruses. PLoS Pathog 6:e 1001005
Lee Y-M, Tscherne D M, Yun S-I, Frolov I, Rice C M. (2005). Dual mechanisms of pestiviral superinfection exclusion at entry and RNA replication. J Virol 79:3231-3242
Lee R F, Keremane M L. (2013). Mild strain cross protection of tristeza: a review of research to protect against decline on sour orange in Florida. Front Microbiol 4: 259.
Lole S, Bollinger R C, Paranjape R S, Gadkari D, Kulkarni S S, Novak N G, et al. (1999) Full-length human immunodeficiencey virus Type 1 genomes from subtype C-infected seroconverters in India, with evidence of intersubtype recombination. J Virol 73: 152-160.
Martin S J, Highfield A C, Brettell L, Villalobos E M, Budge G E, Powell M et al. (2012). Global honey bee viral landscape altered by a parasitic mite. Science 336: 1304-1306.
Mondet F, de Miranda J R, Kretzschmar A, Le Conte Y, Mercer A R. (2014). On the front line: quantitative virus dynamics in honeybee (Apis mellifera L.) colonies along a new expansion front of the parasite Varroa destructor. PLoS Pathog 10:e1004323.
Moore J, Jironkin A, Chandler D, Burroughs N, Evans D J, Ryabov E V. (2011). Recombinants between Deformed Wing Virus and Varroa destructor virus-1 may prevail in Varroa destructor-infested honeybee colonies. J Gen Virol 92: 156-161.
Mordecai G J, Wilfert L, Martin S J, Jones I M, Schroeder D C. Diversity in a honey bee pathogen: first report of a third master variant of the Deformed Wing Virus quasispecies. ISMEJ (2015) 1-10.
Ongus J R, Peters D, Bonmatin J-M, Bengsch E, Vlak J M, van Oers M M. (2004). Complete sequence of a picorna-like virus of the genus Iflavirus replicating in the mite Varroa destructor. J Gen Virol 85: 3747-3755.
R Core Team (2015). R: A language and environment for statistical computing. R foundation for statistical computing: Vienna, Austria.
Ratnieks F L W, Carreck N L. (2010). Clarity on honey bee collapse? Science 327: 152-153.
Ryabov E V, Wood G R, Fannon J M, Moore J D, Bull J C, Chandler D et al. (2014). A Virulent Strain of Deformed Wing Virus (DWV) of honeybees (Apis mellifera) prevails after Varroa destructor-mediated, or in vitro, transmission. PLoS Pathog 10:e1004230.
Salaman R N. (1933). Protective inoculation against a plant virus. Nature 131:468-468.
Schroeder D C, Martin S J. (2012). Deformed wing virus: The main suspect in unexplained honeybee deaths worldwide. Virulence 3: 589-591.
Seeley T D. (2007). Honey bees of the Arnot Forest: a population of feral colonies persisting with Varroa destructor in the northeastern United States. Apidologie 38: 19-29.
Sims D, Sudbery I, Ilott N E, Heger A, Ponting C P. (2014). Sequencing depth and coverage: key considerations in genomic analyses. Nat Rev Genet 15:121-132.
Strauss U, Human H, Gauthier L, Crewe R M, Dietemann V, Pirk C W W. (2013). Seasonal prevalence of pathogens and parasites in the savannah honeybee (Apis mellifera scutellata). J Invertebr Pathol 114: 45-52.
Sumpter D J T, Martin S J. (2004). The dynamics of virus epidemics in Varroa-infested honey bee colonies. J Anim Ecol 73: 51-63.
Thompson C E, Biesmeijer J C, Allnutt T R, Pietravalle S, Budge G E. (2014). Parasite pressures on feral honey bees Apis mellifera sp. PLoS ONE 9: e105164.
Tscherne D M, Evans M J, von Hahn T, Jones C T, Stamataki Z, McKeating J A et al. (2007). Superinfection exclusion in cells infected with Hepatitis C Virus. J Virol 81:3693-3703.
Yang X, Charlebois P, Gnerre S, Coote M G, Lennon N J, Levin J Z et al. (2012). De novo assembly of highly diverse viral populations. BMC Genomics 13:475
Yue C, Genersch E. (2005). RT-PCR analysis of Deformed Wing Virus in honeybees (Apis mellifera) and mites (Varroa destructor). J Gen Virol 86: 3419-3424.
Zioni N, Soroker V, Chejanovsky N. (2011). Replication of Varroa destructor virus 1 (VDV-1) and a Varroa destructor virus 1-Deformed Wing Virus recombinant (VDV-1-DWV) in the head of the honey bee. Virology 417: 106-112.

1-37. (canceled)

38. A method of preventing viral infection of a hymenopterous insect of the superfamily Apoidea comprising the application of an avirulent virus form thereto.

39. A method as claimed in claim 38, in which the method is a method of preventing viral infection of a bee by a virulent virus variant comprising inoculating with an avirulent virus form.

40. A method as claimed in claim 38, in which the method is a method of superinfection exclusion protection of bees comprising inoculation with an avirulent virus form.

41. A method as claimed in claim 38, in which the insect is from the group selected from: the family Apidae; the genus Apis; the genus Bombus; the species Apis mellifera.

42. A method as claimed in claim 38, in which the viral infection prevented is DWV.

43. A method as claimed in claim 38, in which the viral infection prevented is type A DWV.

44. A method as claimed in claim 38, in which the avirulent virus is naturally occurring and/or recombinant and/or mutated or variant forms thereof.

45. A method as claimed in claim 38, in which the avirulent virus is type B DWV.

46. A method as claimed in claim 38, in which the avirulent virus form is type C DWV.

47. A method as claimed in claim 38, in which the avirulent form is administered by: topical application; immersion or spraying; contamination; or through a food.

48. A method as claimed in claim 47, in which the food is natural.

49. A method as claimed in claim 47, in which the food is synthetic.

50. A method according to claim 38, in which the avirulent form is administered by: injection; by introduction of a vector; by introduction or infected mites; by introduction of infected Varroa destructor mites.

51. An inoculum formulation comprising an effective dose of an infectious avirulent viral form and a suitable carrier or excipient.

52. An inoculum formulation as claimed in claim 51, comprising an effective dose of infectious type B DWV or a variant or mutant thereof and a suitable carrier or excipient.

53. A method of preventing infection of Apis mellifera by type A DWV comprising inoculating with type B DWV or a variant or mutant form thereof.
